(19) United States
(12) Patent Application Publication
(10) Pub. No.: US 2013/0152930 A1
(43) Pub. Date: Jun. 20, 2013

(54) COLD CLIMATE AIR EXCHANGER

(71) Applicants: Thomas Votel, Sunfish Lake, MN (US); Adria Hardy, Columbia Heights, MN (US); Jeffrey A. Popowski, Roseville, MN (US)

(72) Inventors: Thomas Votel, Sunfish Lake, MN (US); Adria Hardy, Columbia Heights, MN (US); Jeffrey A. Popowski, Roseville, MN (US)

(21) Appl. No.: 13/690,622

(22) Filed: Nov. 30, 2012

Related U.S. Application Data
(60) Provisional application No. 61/577,853, filed on Dec. 20, 2011.

(51) Int. Cl. A61M 16/10

(52) U.S. Cl. CPC A61M 16/1075
USPC 128/204.17

(57) ABSTRACT

The present disclosure is directed to a novel cold climate air exchanger. In one aspect of the disclosure, the cold climate air exchanger is comprised of an interior and an exterior panel that houses an open-celled foam filter insert there between. The assembly cooperates to gain a light weight air exchanger that advantageously regulates the natural moisture and temperature of the inhaled and exhaled air while maintaining adequate airflow for the user.

### CLAIM OF BENEFIT TO PRIOR APPLICATIONS

This application claims the benefit of U.S. Provisional Application No. 61/577,853 filed Dec. 20, 2011; the entirety of which is hereby fully and completely incorporated herein by reference.

### FIELD

The present invention relates to a cold climate air exchanger for use in cold environments. More particularly the invention relates to an air exchanger for use in cold weather clothing such as a balaclava, scarf or mask.

### BACKGROUND

Under normal breathing conditions a person inhales around ½ liter of air per breath. When breathing in air temperatures that are low, the coldness and dryness of the air can cause the muscles around the airway to tighten as the body attempts to restrict the flow of the frigid air into the lungs. This airway narrowing is sometimes referred to as exercise asthma, or cold air-induced asthma, and restricts the normal volume of air retrieved in an inhalation. If a person already has a lung disease, or a condition like asthma, the effect of the cold air on the lungs can further exacerbate breathing difficulties. This may lead to even more restricted airflow into the lungs.

Under such conditions it is advantageous to warm the air before it enters the lungs. The prior art cold air exchangers directed to this task have attempted to perform this function, but have failed to adequately manage airflow, control the natural moisture of the exhaled air, and otherwise have failed to properly control the environment of the mask's interior. One undesirable result of the prior art is that the mouth covering apparatus does not adequately warm the incoming air or retain the natural moisture of the air being expelled by the lungs. Further the prior art devices undesirably retain too much natural moisture. In the former case, the dry air can still cause the restricting of the airway, and in the latter case excess moisture can build up inside the mask causing discomfort for the wearer, causing the opening in the mask to freeze over, further restricting the ability to breath.

For at least these above-described reasons there is a need for an improved cold weather air exchanger that will warm the incoming air, control the natural moisture of the exhaled air to allow it to be re-circulated without excess moisture buildup within the assembly, and not restrict the normal volume of air required by the user.

### SUMMARY

The present disclosure is directed to a cold climate air exchanger that warms cold air before it enters the lungs, and advantageously controls the natural moisture content of the exhaled air to discourage freezing around and in the assembly.

The above summary is not intended to limit the scope of the invention, or describe each embodiment, implementation, feature or advantage of the invention.

### BRIEF DESCRIPTION OF THE DRAWINGS

FIG. 1 is a perspective view of the present invention.

FIG. 2 is a front elevation view of the exterior panel.

FIG. 3 is a rear elevation view of the interior panel.

FIG. 4 is a perspective analytic view of the present invention.

FIG. 5 is a front elevation view of a filter element.

FIG. 6 is a front elevation view of the invention incorporated in a balaclava.

### DETAILED DESCRIPTION

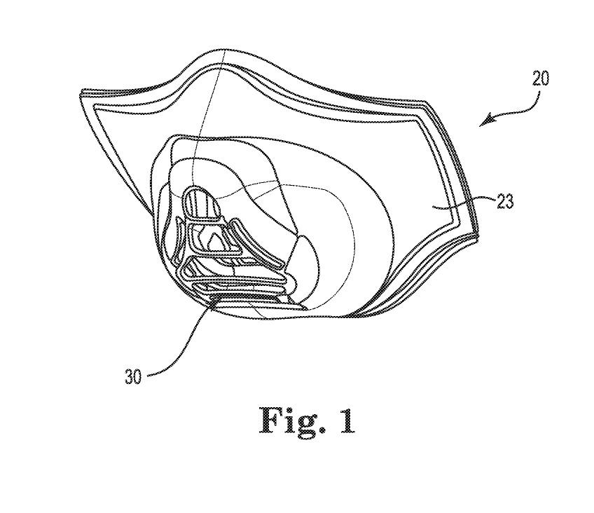

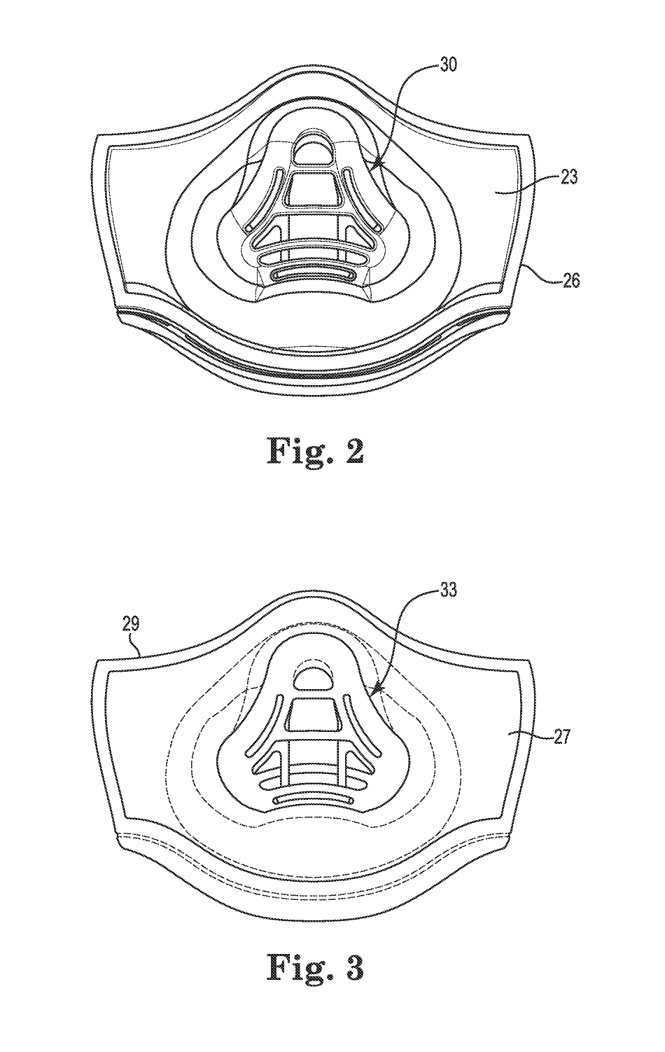

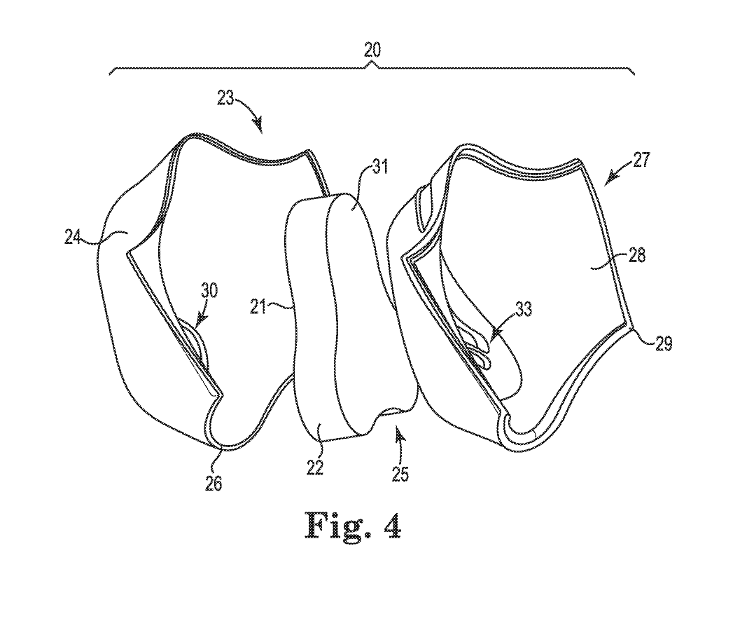

FIGS. 1-3 depict views of a preferred embodiment of the invention. The cold climate air exchanger 20 is preferably cup-shaped to sealably fit over the nose, around the mouth and down to the chin area of a user. The preferred embodiment is comprised of exterior panel 23, filter 25 (not visible, see FIG. 4) and interior panel 27.

Preferably, the seal between the user's nose and interior panel 27 is more air tight than the seal around the lower chin area, allowing for exiting excess airflow away from the face and eyes to reduce fogging effects if the user is wearing glasses or goggles. Also, indirect drainage may be provided out of the bottom of the mask without the need for physical holes apart from openings 30 and 33.

It is anticipated that openings 30 and 33 could be sized depending on the intended use of the invention by a user. A larger opening 30 and 33 would accommodate a greater air-flow requirement and would better suit a larger person, or a person working under more physically demanding conditions. Smaller openings 30 and 33 might better accommodate a smaller user or one with a less demanding air-flow requirement.

FIG. 4 is an analytic perspective view of cold climate air exchanger 20. In a preferred embodiment filter 25 lays between exterior panel 23 and interior panel 27. Exterior or front panel 23 has an outer perimeter 26 and interior or rear panel 27 has an outer perimeter 29. The opening 33 in the interior panel and opening 30 in the exterior panel are preferably generally aligned, when air exchanger 20 is assembled, to expose between about 75% to 95% of the front and rear surface of filter 25 to air flow.

Temperature and moisture management is assisted and gained by the construction and arrangement of the interior panel 27, exterior panel 23 and filter 25. Filter 25 is preferably comprised of an expanded open-cell foam material. The external panel 23 and interior panel 27 are preferably and advantageously fabric covered being comprised of non-porous closed cell foam materials. The exterior portion 24 of front panel 23 is preferably covered with a fabric of cross-linked polyethylene. The interior surface 28 of the back panel 27, that contacts the users face, is preferably covered with a lyra type fabric. This configuration is less prone to transmit cold from the outside of the mask to the interior, as would rubber or plastic. The configuration also resists condensation accumulation on the external and internal portions of the invention. These materials and their configuration also provide for a lighter weight, greater durability, an ergonomic fit with better comfort levels when wore for long durations. These materials also resist the effects of exposure to harsh environments, and promote washability.

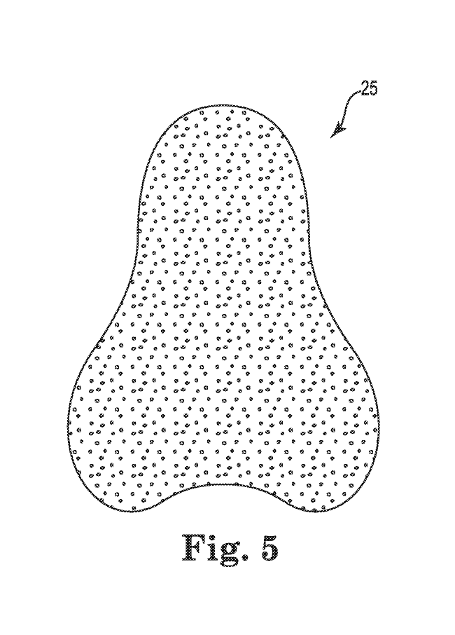

The exemplary filter of FIG. 5 is preferably comprised of open-cell foam that is preferably of a polyether polyurethane with a density between 1.75 and 2.25 PCF (Pounds Per cubic Foot) and a PPI (Pores Per Inch) count of between 18 and 22. The thickness 22 of the open-cell foam material is preferably between ½ inch and 1 inch thick. The filter has a front surface 21 and a rear surface 31 and is between 3 and 6 square inches in total surface area.

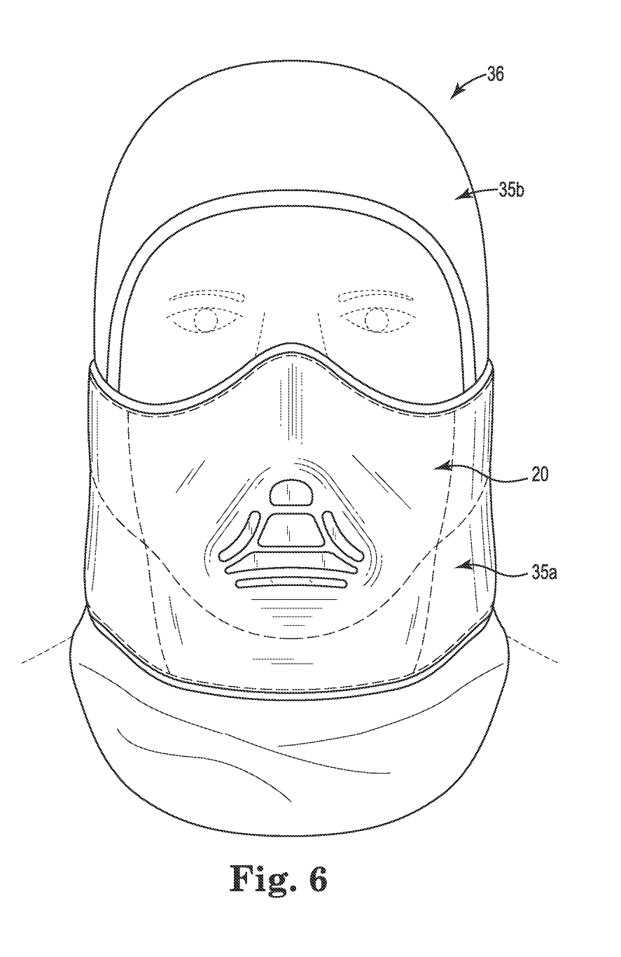

FIG. 6 depicts an alternate embodiment wherein the cold climate air exchanger 20 is incorporated into a balaclava 36. It is anticipated that the cold climate air exchanger may be incorporated into a scarf portion 35a, that could removably attach to the head portion 35b of balaclava 36. Alternatively the cold climate air exchanger could be incorporated into a one piece balaclava, or a mask.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. A cold climate air exchanger comprising:
a front panel of non-porous material having a perimeter and defining an opening generally at a center of the front panel;
a rear panel of non-porous material having a perimeter and defining an opening generally at a center of the rear panel;
a porous filter having a density of between 1.75 and 2.25 PCF;
wherein the front panel and rear panel are joined at their perimeters such that the opening of the front panel is generally aligned with the opening of the rear panel and such that a cavity is defined between the front panel and the rear panel; and
wherein the porous filter is disposed within the cavity and adjacent the generally aligned openings

2. The cold climate air exchanger of claim 1 wherein the filter is comprised of a polyether polyurethane.

3. The cold climate air exchanger of claim 1 wherein the filter is between ½ inch and 1 inch in thickness.

4. The cold climate air exchanger of claim 1 wherein the filter has between 18 and 22 pores per square inch.

5. A cold climate air exchanger comprising:
a front panel having a cup shape and being comprised of a non-porous fabric, the front panel having a perimeter and defining an opening generally at the apex of the front panel;
a rear panel having a cup shape and being comprised of a non-porous fabric, the rear panel having a perimeter and defining an opening generally at the apex of the rear panel;
a porous filter;
wherein the front panel and rear panel are joined at their perimeters such that the opening of the front panel is generally aligned with the opening of the rear panel, and such that a cavity is defined between the front panel and the rear panel; and,
wherein the porous filter is disposed within the cavity and adjacent the generally aligned openings.

6. The cold climate air exchanger of claim 5 wherein the cold climate air exchanger is incorporated into a balaclava.

7. The cold climate air exchanger of claim 5 wherein the cold climate air exchanger is incorporated into a scarf.

8. The cold climate air exchanger of claim 5 wherein the cold climate air exchanger is incorporated into a mask.

9. The cold climate air exchanger of claim 5 wherein the filter has a front surface, a rear surface and a thickness of between ½ inch and 1 inch;
wherein the front panel opening exposes between about 75% and 95% of the front surface of the filter; and,
wherein the rear panel opening exposes between about 75% and 95% of the rear surface of the filter.

10. A balaclava containing a cold climate air exchanger comprising:
a cup shaped portion of non-porous material having a front side and a rear side and defining a volume there between, the cup shaped portion further defining a front side opening located generally at the center of the front side, and a rear side opening located generally at the center of the rear side, said openings being generally aligned;
a porous filter, having a front surface area, a thickness and a rear surface area, the porous filter being disposed within the volume;
wherein the filter covers the generally aligned openings; and,
wherein the cup shaped portion is comprised of fabric and is incorporated into a portion of a balaclava.

11. The cold climate air exchanger of claim 8 wherein the filter has a density of between 1.75 and 2.25 PCF and contains between 18 and 22 pores per square inch.

12. The cold climate air exchanger of claim 8 wherein the filter is between ½ inch and 1 inch thick and the generally aligned openings expose between about 75% and 95% of the front and rear surface areas of the filter.
